SEQUENCE LISTING (11 sequences)

SEQ ID NO: 1 - DNA, 43 bp, Homo sapiens
gatatagcagccctggtgtagtttcttcatttcaggaagactg

SEQ ID NO: 2 - DNA, 21 bp, Artificial Sequence
  Description of Artificial Sequence Primer
gaaggtcggagtcaacggatt

SEQ ID NO: 3 - DNA, 23 bp, Artificial Sequence
  Description of Artificial Sequence Primer
ggatctcgctcctggaagatggt

SEQ ID NO: 4 - DNA, 25 bp, Artificial Sequence
  Description of Artificial Sequence Primer
gggccactgtcggcatcatgattgg

SEQ ID NO: 5 - DNA, 32 bp, Artificial Sequence
  Description of Artificial Sequence Primer
tgtagctgttgcaaatgctttaaggaagaagc

SEQ ID NO: 6 - DNA, 21 bp, Artificial Sequence
  Description of Artificial Sequence Primer
cagacacacggtgaactatgg

SEQ ID NO: 7 - DNA, 22 bp, Artificial Sequence
  Description of Artificial Sequence Primer
gatcagcttccactgttagacg

SEQ ID NO: 8 - DNA, 46 bp, Homo sapiens
gggccactgtcggcatcatgattggtgtactggctggagttactct

SEQ ID NO: 9 - DNA, 46 bp, Homo sapiens
gggccactgtcggcatcatgattggagtgctggttggggttgctct

SEQ ID NO: 10 - DNA, 106 bp, Homo sapiens
ttattatatcaaactcttatcagagttttagcaatagtcattttacatgatggtcattcacagatagttgttttgcttcttccttaaagcatttgcaacagctaca

SEQ ID NO: 11 - DNA, 106 bp, Homo sapiens
ttattataataaattatcatcagatttttaactgtactcattttaaatctttgtcattcacagacagttgttttgcttcttccttaaagcatttgcaacagctaca

(19) United States
(12) Patent Application Publication
(10) Pub. No.: US 2006/0019250 A1
(43) Pub. Date: Jan. 26, 2006

(54) Method for the detection of carcinoembryonic agent and cancer diagnosis method based thereon

(75) Inventors: Hartmut Juhl, Hamburg (DE); Regina Hampton, Washington, DC (US)

Correspondence Address: PILLSBURY WINTHROP SHAW PITTMAN, LLP, P.O. BOX 10500, MCLEAN, VA, 22102, US

(73) Assignee: Georgetown University, Washington, DC (US)

(21) Appl. No.: 10/504,354

(22) PCT Filed: Feb. 13, 2003
(86) PCT No.: PCT/US03/04201

§ 371 Date: Jul. 6, 2005

Related U.S. Application Data
(60) Provisional application No. 60/355,834, filed on Feb. 13, 2002.

(51) Int. Cl. C12Q 1/68; C07H 21/04; C12P 21/06; C07K 14/82

(52) U.S. Cl. 435/6; 435/69.1; 435/320.1; 435/325; 530/350; 536/23.2

(57) ABSTRACT

The present invention provides a method of detecting carcinoembryonic antigen (CEA) comprising detecting a DNA sequence comprising the sequence: GATATAGCAGCCCTGGTGTAGTTTCTTCATTTCAGGAAGACTG. The invention also provides a method of diagnosis of disseminated cancer comprising obtaining a cell sample, extracting DNA from the cell and detecting the presence of the sequence: GATATAGCAGCCCTGGTTSGTTCTTCATTTCAGGAAGACTG.

### CROSS-REFERENCE TO RELATED APPLICATION

This application claims priority from U.S. Provisional Application Ser. No. 60/355,834, filed Feb. 13, 2002, which is incorporated herein in its entirety.

### FIELD OF THE INVENTION

The present invention relates to a method of detection of carcinoembryonic agent (CEA) and cancer diagnosis method.

### SUMMARY OF THE RELATED ART

Colorectal cancer is the third most common malignancy in the United States, with an incidence of 160,000 new patients per year. Only 40% to 50% of the patients survive longer than 5 years (1). Mortality is due to metastatic disease which occurs most often in the liver, followed by the lung. A chance of cure depends on complete surgical removal of the tumor.

5-Fluorouracil (5-FU) is the first line drug for chemotherapy and shows 20-30% response rates in metastatic patients but rarely achieves cure (2).

Numerous clinical studies indicate that carcinoembryonic antigen (CEA) promotes metastatic growth of colon cancer (3). High preoperative CEA serum levels correlate with a poor clinical outcome in colorectal (4), gastric (5), lung (6) and breast cancer (7). Loss of apical CEA expression and diffuse cytoplasmic staining of CEA in colon cancer is also associated with metastatic disease (8) as is CEA expression by circulating colon cancer cells (9). However, although these clinical data strongly suggest a role for CEA in the progression of colon cancer and possibly other malignancies, experimental studies have failed to conclusively determine the biological role of CEA.

CEA was first described as an oncofetal antigen in 1965 (10) and is overexpressed in a majority of carcinomas including cancer of the colon, breast and lung. It is a glycoprotein of approximately 180 kDa, belongs to the immunoglobulin supergene family and is anchored in the cell membrane via a glycosyl phosphatidyl inositol moiety (11).

Overall, the data are conflicting regarding the function of CEA in cancer models. Marked dysregulation of the expression of CEA subgroup members has been noted in colorectal cancer (12) and their differential expression may be important in pathobiochemistry and biology of CEA-positive cancers. Some authors suggest that CEA is a homophilic and heterophilic adhesion molecule (13-16) which may also stimulates release of prometastatic cytokines by Kupffer cells in the liver (17, 18). Other studies propose that CEA serves as a repulsion molecule which increases the mobility of tumor cells (19) but may also function as an immune escape mechanism (20). Because the data from these studies are derived from studying effects of nonphysiologically high levels of CEA, the significance of CEA in metastatic growth has been questioned (22).

The main limitation of current cancer therapy is the development of metastatic disease which, in most patients, is not curable. In colon cancer, which counts for approximately ⅓ of all cancer related deaths in the US, cure depends on radical surgery prior the development of metastases. However, even after an apparent curative surgical intervention approximately 50% of the patients experience metastatic relapse that in most cases eventually leads to death.

Dissemination of tumor cells is an obligatory step toward progression to metastatic disease. In the 1950's, cytological studies demonstrated that circulating cancer cells from solid tumors are detectable in the blood of cancer patients (33) In the 80's and 90's highly sophisticated techniques were developed, such as immunocytology (34-36) and RT-PCR (37-39) enabling detection of circulating colon cancer cells in various body compartments. In colon cancer finding of minimal residual disease in bone marrow, peritoneal lavage and lymph node samples correlated with poor survival (40, 41).

Detection and quantification of circulating cancer cells in whole blood samples could serve as unique and easy diagnostic tool to determine prognosis and therapeutic effectiveness of cancer therapy. So far, only RT-PCR has been shown to provide the sensitivity and practicability necessary to detect the low numbers of colon cancer cells present in whole blood samples.

### SUMMARY OF THE INVENTION

The invention is based on the unexpected discovery of a splice in the nucleic acid sequence of CEA which interferes with the detection of “non spliced” CEA in cancer cells. The splice is situated at a region including the nucleotides from nucleotide 2199 to nucleotide 2241 of the sequence of CEA as described in the PubMed NIH library with Accession=M17303, NID=g178676. Those nucleotides correspond to the sequence: GATATAGCAGCCCTGGTGTAGTTTCTTCATTTCAGGAAGACTG. The corresponding nucleotides in the spliced CEA, which were unkown prior to the subject invention are shown in FIG. 4C.

The present invention provides a method of detecting carcinoembryonic antigen (CEA) comprising detecting a DNA sequence comprising the sequence: GATATAGCAGCCCTGGTGTAGTTTCTTCATTTCAGGAAGACTG. The invention also provides a method of diagnosis of disseminated cancer comprising obtaining a cell sample, extracting DNA from the cell and detecting the presence of the sequence:

GATATAGCAGCCCTGGTGTAGTTTCTTCATTTCAGGAAGACTG.

The invention further provides an isolated nucleic acid molecule comprising a polynucleotide selected from the group consisting of:
(a) the sequence defined by nucleotide 49 to nucleotide 108 contained in FIG. 4C;
(b) the sequence defined by nucleotide 50 to nucleotide 107 contained in FIG. 4C;
(c) the polynucleotide complement of the polynucleotide of (a) or (b); and
(d) a polynucleotide at least 90% identical to the polynucleotide of (a), (b) or (c) and a polypeptide encoded by the polynucleotide.

To quantify circulating colon cancer cells in whole blood samples, we established real-time RT-PCR using carcinoembryonic antigen (CEA) and cytokeratin 20 (CK20) as target molecules and compared patients with metastatic disease (n=32) and healthy volunteers (n=17). CK20 mean values were elevated in cancer patients (p<0.001) and defined a subgroup (36%) who showed CK20 levels at least 100-fold above the highest value of the healthy control group. In contrast, only two patients (6%) showed elevated CEA levels (p>0.05). While melting curve analysis of tumor cells and CEA cDNA results in a PCR product of 82° C. melting temperature, samples of the control group revealed a CEA-PCR product of 79° C. However, 30% of the cancer patients expressed in addition the “correct” 82° C. product. Sequence analysis indicate the presence of a CEA splice variant in white blood cells.

### BRIEF DESCRIPTION OF THE DRAWINGS

Table 1: (A) shows the primer sequences for GAPDH, CEA and CK20 and the length and melting point of the respective PCR products as determined in human HT29 and LS147T colon cancer cells.
(B) The table summarizes the set-up of the PCR cycler for GAPDH, CEA and CK20 PCR.

FIG. 1: Representative data for CEA (A), CK20 (B) and GAPDH (C) standard curves which were used to quantify the PCR products of clinical samples. (D) shows the melting curve for the respective PCR products. As standard for CEA (A) a bluescript vector containing the CEA full length cDNA and for CK20 (B) total RNA of HT29 colon cancer cells were diluted in 10-fold steps as indicated. The GAPDH standard (C) represents copy numbers as provided by the manufacturer of the kit. The orange line defines the threshold level.

FIG. 2: Evaluation of CEA (A) and CK20 (B) levels in 32 metastatic colon cancer patients (left) and blood samples from 17 healthy volunteers (right). The bar indicates mean values. CK20 values differed significantly between cancer patients and the control group (p<0.001).

FIG. 3: Evaluation of the melting curve for the CEA real time RT-PCR.
(A) shows data of a representative experiment which included 7 samples of the healthy control group (upper panel) and 8 samples of colon cancer patients. In cancer patients a second peak at 82° C. appeared.
(B) Spiking experiment of human LS147T colon cancer cells in whole blood of a healthy volunteer. Different amounts of cells were mixed with 10 ml whole blood and processed by real-time RT-PCR. The upper three panels demonstrate the melting curve analysis of 0-10² tumor cells (upper panel), 10³-10⁵ cells (middle panel) and 10⁶-_ 10⁷ cells (lower panel). At the bottom the melting curve of the CEA full length cDNA standard is shown. The melting point of 82° C. is identical to the melting point found in human colon cancer cells.

FIG. 4: This figure illustrates the structure of the CEA gene (A) and the corresponding PCR products as found in human colon cancer cells (B) and in white blood cells (C). In addition, the sequence of the CEA product of white blood cells is shown (upper lane of nucleotides). Homology to the respective gene sequence (lower lane of nucleotides) as found in the NCBI data base (GI #15281189) is indicated.

### DETAILED DESCRIPTION OF THE PREFERRED EMBODIMENTS

In this study we evaluate CEA and CK20 as target molecules for the detection and quantification of circulating cancer cells by real-time RT-PCR because these are frequently used markers which have been successfully applied by several independent research groups (37, 38, 42-44). However, amplification has been reported in normal samples by using high cycle numbers indicating broad expression of these markers at low levels (43, 45, 46). Quantification of the PCR signal would allow calculation of cell numbers. It would also solve problems arising from background signals by defining cut-off values in cancer patients.

We analyzed the sensitivity and reliability of the Amplifluor®—and the SYBR®—Green approach. Because significant differences between patients with metastatic colon cancer and healthy volunteers are mandatory to assume any clinical benefit, we focused this clinical pilot study on these two groups.

Detection and quantification of circulating cancer cells in whole blood samples could serve as unique and easy diagnostic tool to determine prognosis and therapeutic effectiveness of cancer therapy. So far, only RT-PCR has been shown to provide the sensitivity and practicability necessary to detect the low numbers of colon cancer cells present in whole blood samples.

In this study we evaluate CEA and CK20 as target molecules for the detection and quantification of circulating cancer cells by real-time RT-PCR because these are frequently used markers which have been successfully applied by several independent research groups. However, amplification has been reported in normal samples by using high cycle numbers indicating broad expression of these markers at low levels. Quantification of the PCR signal would allow calculation of cell numbers. It would also solve problems arising from background signals by defining cut-off values in cancer patients.

We analyzed the sensitivity and reliability of the Amplifluor®—and the SYBR®—Green approach. Because significant differences between patients with metastatic colon cancer and healthy volunteers are mandatory to assume any clinical benefit, we focused this clinical pilot study on these two groups.

Material and Methods

Cell Lines. Human HT29 and LS147T colon cancer cells were obtained from American Type Culture Collection (ATCC, Rockville, Md.) and were maintained in continuous culture at 37° C./5% CO₂ using modified IMEM (Life Technologies Inc., Gaithersburg, Md.) supplemented with 1% glutamine and 10% heat-inactivated fetal bovine serum (FBS).

Samples. Following IRB approval by the board of Georgetown University, whole blood was obtained from 32 patients who were treated for metastatic colon cancer. In addition, we analyzed blood samples from 17 healthy volunteers. The first 5 ml of blood was discarded to avoid contamination with skin cells. 20 ml of blood was collected in Vacutainer tubes with K₃ EDTA (Becton Dickinson-Franklin Lakes, N.J.).

Mononuclear cells were separated using Ficoll-Paque Plus (Amersham, Piscataway, N.J.) density centrifugation as described (4). Cells were resuspended in RNA-STAT60 (Tel-Test, Friendswood, Tex.) and total RNA was prepared according to the manufactures manual modified by the use of 30 mg glycogen during isopropanol precipitation at −20° C. Preparation of cDNA was performed as described (37) using random hexamer primer (PE Applied Biosystems, Foster City, Calif.).

Primer and standard. Table 1a gives an overview about the primer sequences used in the PCR. Amplification of CEA was performed using previously published sequences, i.e. for the nested approach primer pair A/B and primer pair C/B, respectively (37). With respect to the Amplifluor®-method, the required Z-sequence (see protocol of manufacturer) was attached to the C-primer. CK20 PCR-amplification was performed using primers and protocol published by Burchill et al. (38). As standard for CEA we used full length CEA cDNA ligated into the bluescript vector (Invitrogen) which was kindly provided by Dr. M. Neumaier, Dept. of Clinical Chemistry, Hamburg, Germany. As a CK20 standard we prepared a RNA stock of HT29 colon cancer cells which express high CK20 levels (39). The relative amount of CK20 refers to the total RNA of HT29 cells.

Real-time RT-PCR using Amplifluor®. For CEA, cDNA was directly amplified (2: g total RNA) using primar pair B and C (modified with Z-tail). Alternatively, a nested RT-PCR was performed as previously published (37) with 20 cycles using 2: g total RNA and primer pair A and B preceeding the real-time measurement. The protocol followed the manual of the “Amplifluor Universal Amplification and Detection Kit” (Intergen, Purchase, N.Y.) using Platinum Taq DNA polymerase (Life Technologies, Rockville, Md.). The temperature set up of the PCR reaction is shown in table 1b. Modification to the previously published protocol included an annealing temperature of 60° C. instead of 72° C. which is a requirement of the Kit. Before doing so we confirmed that this had no impact of the specificity of the PCR reaction. For amplification, we used the “iCycler” a real time PCR machine purchased from BioRad (Hercules, Calif.).

Real-time RT-PCR using SYBR®-Green. All reagents except Platinum Taq polymerase (Life Technologies) were obtained from the “SYBR®-Green Core Reagents Kit” (PE/Applied Biosystems).

For CEA we used the primer pair B and C as previously published. A modification of the published protocol (37) included an annealing temperature of 60° C. instead of 72° C. and the use of 40 cycles. The change of the annealing temperature allowed comparison with the Amplifluor® method. Analysis of CEA full length cDNA, colon cancer cells and blood samples showed no differences in specificity between both annealing temperatures.

CK20 RT-PCR followed the protocol published by Burchill et al. (38). Table 1b gives an overview of the PCR conditions.

GAPDH-Real time RT-PCR. To control for the RNA amount in the reaction mix we used GAPDH, a common housekeeping gene. An aliquot of each sample was analyzed in parallel to CEA and CK20 RT-PCR using the “Amplifluor Direct Gene Systems Kit for GAPDH” (Intergen) and Platinum Taq DNA polymerase (Life Technologies). This kit also provides a GAPDH cDNA template to calculate a standard curve. The procedure followed the protocol provided by the manufacturer. The threshold levels obtained from the CEA and CK20 PCR were adjusted to the threshold levels found in the GAPDH reaction to correct for minor variations in RNA loading.

Sequencing. PCR products were directly sequenced in the core facility of the Lombardi Cancer Center using the “Big Dye Terminator Sequencing Reaction Kit” (PE/Applied Biosystems).

Statistical test. For statistical analysis the unpaired t-test was used.

Comparison Amplifluor® and SYBR®-Green for CEA RT-PCR. The Amplifluor® kit requires an annealing temperature not higher than 60° C. Because the CEA RT-PCR was established at annealing temperatures of 72° C. for the B and C primer pair we first compared the Amplifluor® method and SYBR®-Green at both temperatures using CEA full length cDNA, HT29 and LS147T colon cancer cells, and samples from healthy volunteers. The specificity of the PCR reaction did not differ between 60° C. and 72° C. as demonstarted by gel electrophoresis, melting curve analysis and sequencing (data not shown). Both methods allowed reliable quantification of CEA cDNA. However, the sensitivity of the SYBR®-Green approach was approximately 1000-fold higher. The Amplifluor® Kit achieved similar sensitivity only when used in a nested approach, i.e. when 20 cycles using the primer pair A and B preceeded the real time measurement. However, nested RT-PCR hindered the establishment of a reliable standard curve because the preceeding 20 PCR cycles caused a significant variation. Finally, we tested if 40 cycles and the exclusive use of the primer B and C produced a dectable CEA product from patients whole blood samples. While the Amplifluor® kit failed to show a signal, SYBR®-Green achieved reliable signals which were within the range of the CEA cDNA standard curve.

In summary, these data demonstrated that both methods when used as a single PCR (40 cycles) allowed reliable quantification of CEA. However, only SYBR®-Green was sensitive enough to detect signals from whole blood samples. Therefore, we chose this approach for further studies.

Standard curve for CEA and CK20 (SYBR®-Green) and GAPDH (Amplifluor®). Standard curves ranged from 10⁻⁹ g to 10⁻¹⁸ g of CEA cDNA, 10⁻⁴ to 10⁻¹¹ g total RNA of HT29 cells for the detection of CK20 and from 10⁸ to 10¹ copy numbers of GAPDH. Dilution of total RNA from HT29 and LS147T colon cancer cells showed that real time RT-PCR was able to detect reliable signals from one cell (data not shown). The specificity was controlled by melt curve analysis. The purity and length of the product was confirmed by gel electrophoresis and sequencing.

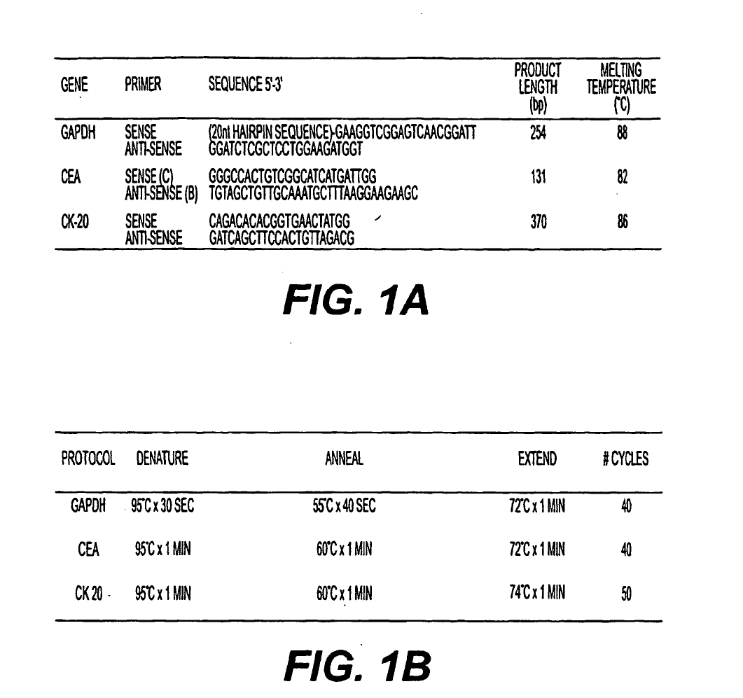

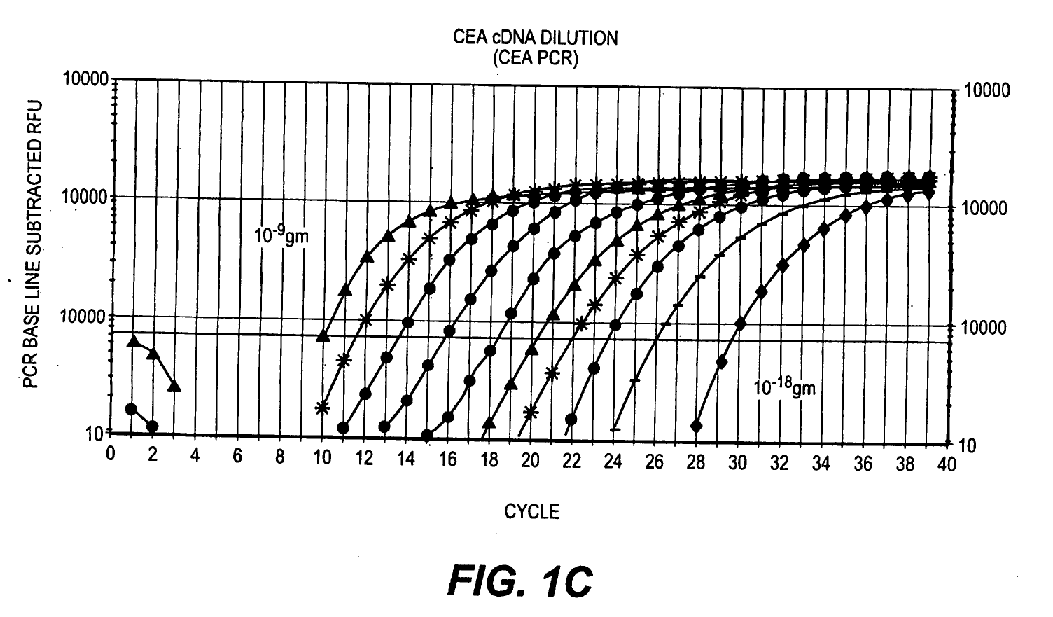

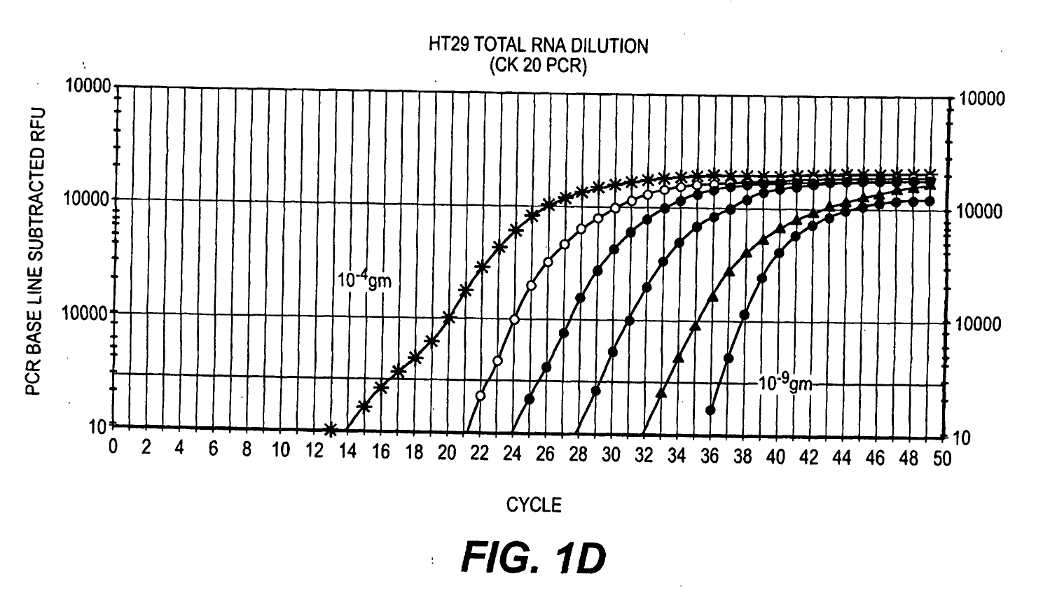

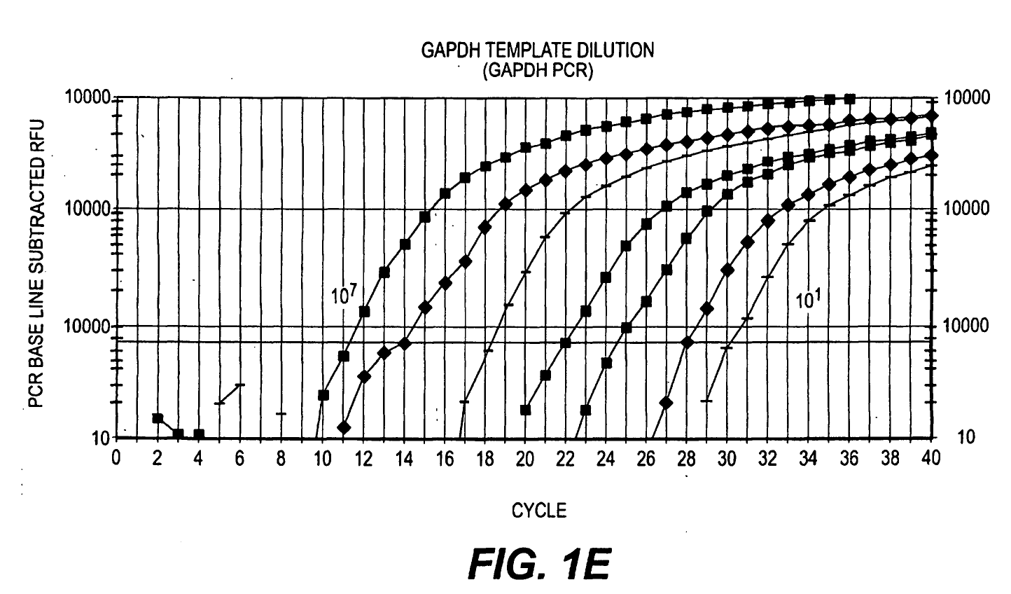

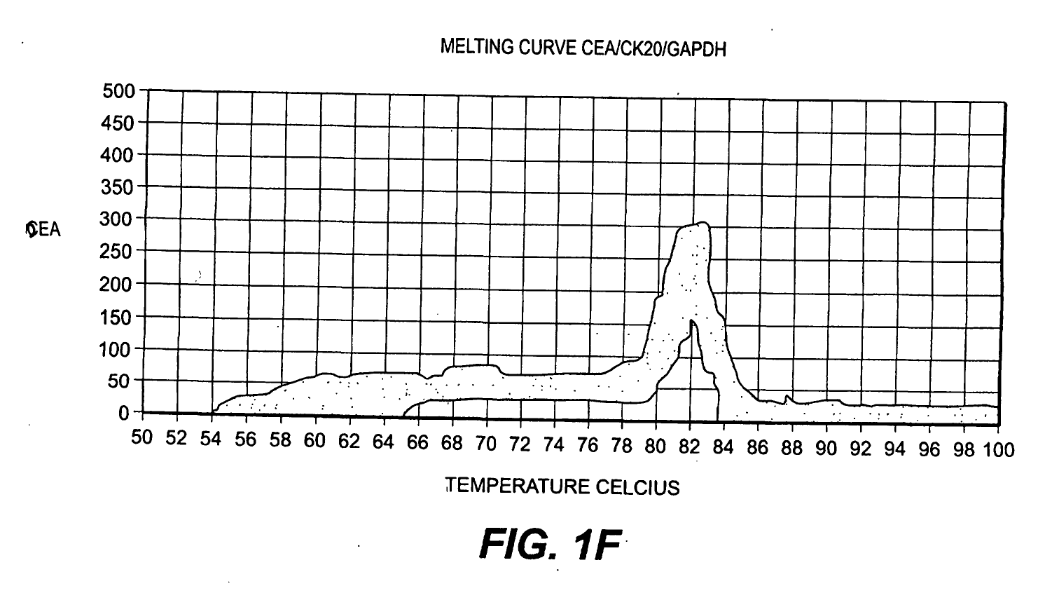

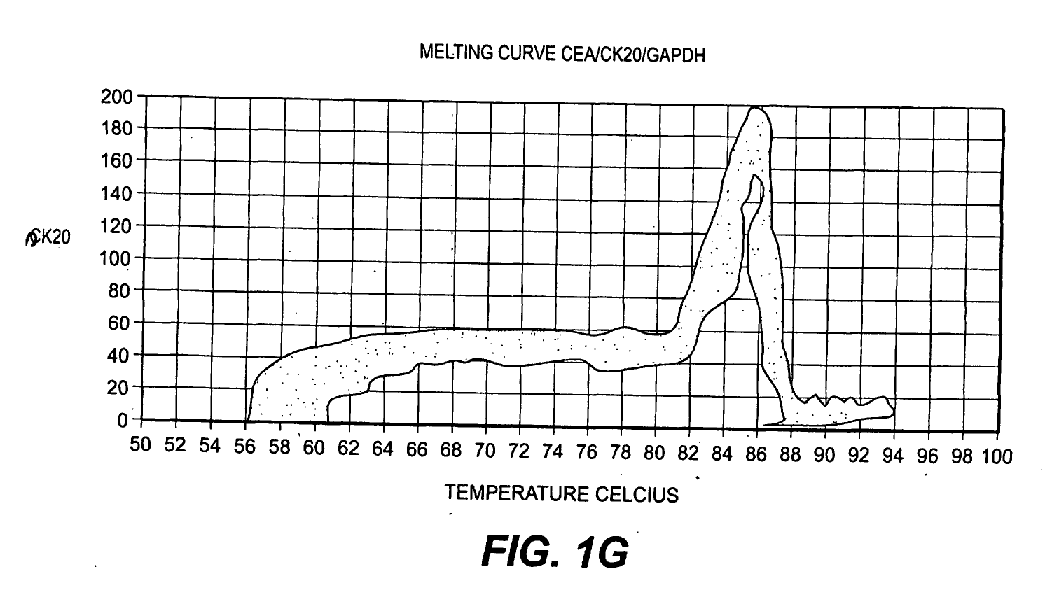

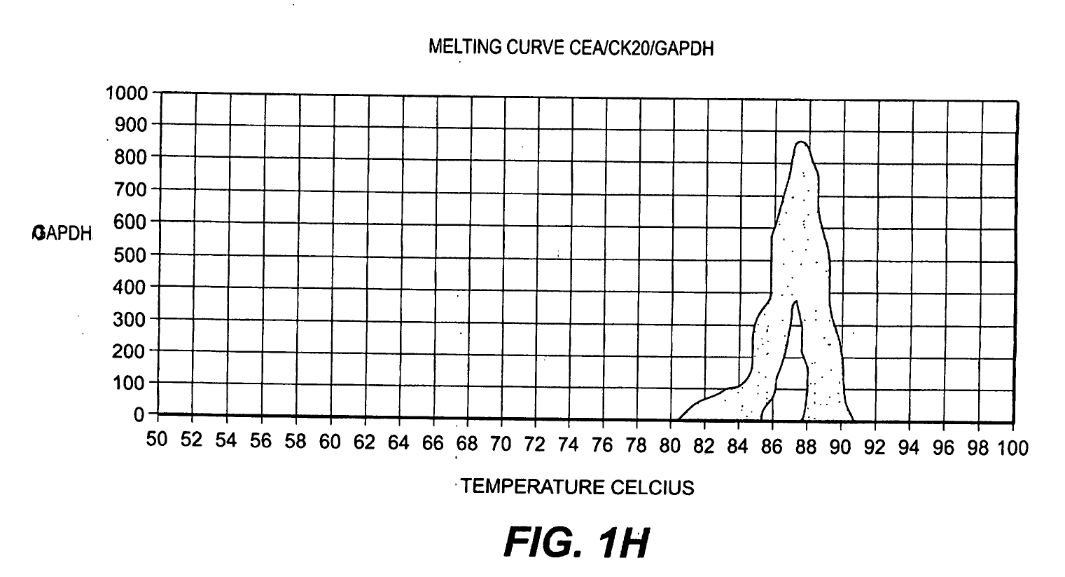

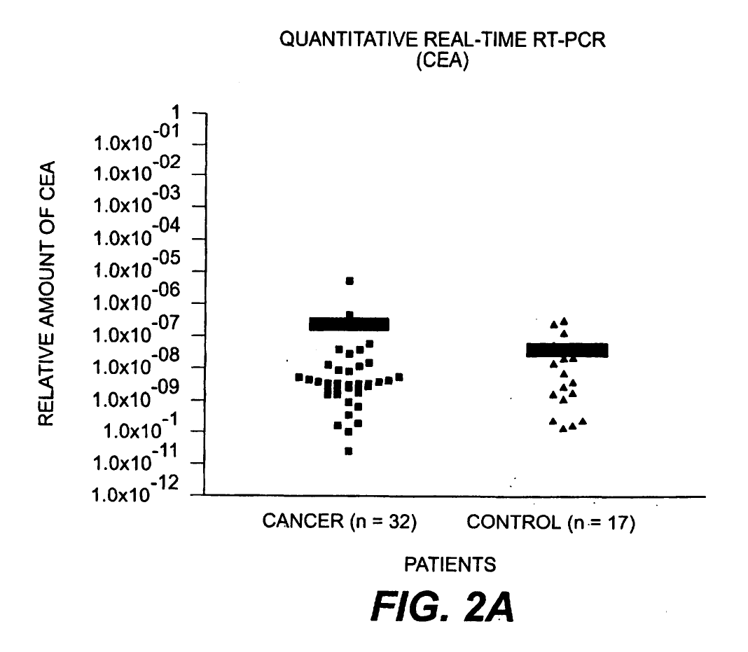

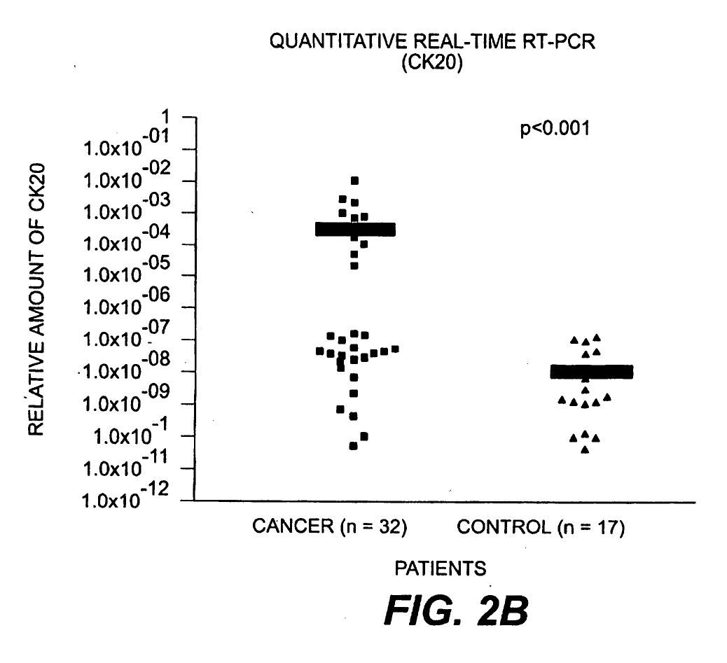

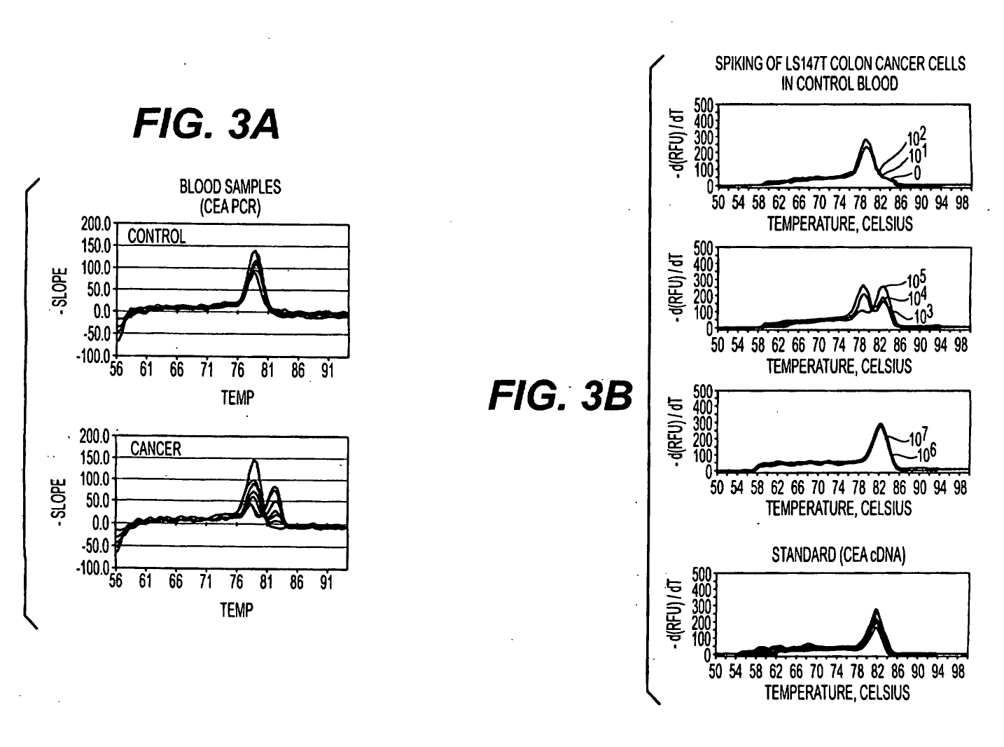

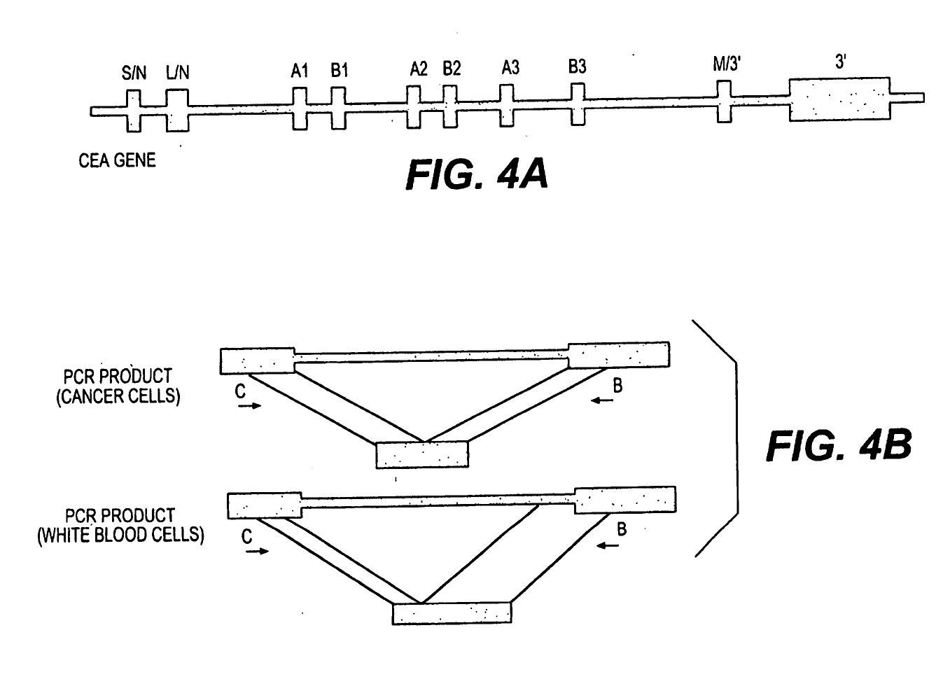

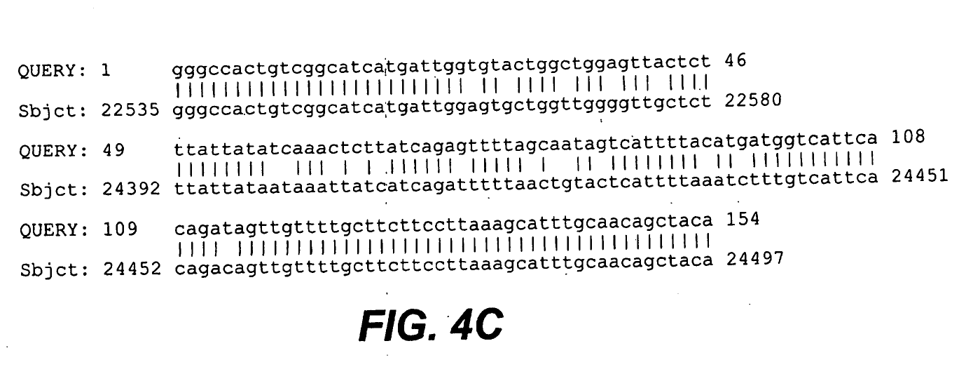

Clinical samples. As shown in FIG. 1 both markers resulted in a significant background signal in the healthy control group suggesting low expression of CEA and CK20 in white blood cells. However, the mean values in cancer patients were 10-fold higher for CEA (p>0.05) and 10,000-fold higher for CK20 (p<0.001). When we took the highest value of the control group as a cut-off level two cancer patients (6%) had elevated CEA values. In contrast, using CK20 as a detection marker 12 patients (36%) of the cancer patients had at least 100-fold higher values (p<0.001). The high CK20 values of nine (75%) of these patients were reflected by high CEA values. Assuming that a single HT29 cancer cell contains approximately 1 pg total RNA and that HT29 colon cancer cells and circulating cancer cells have similar CK20 levels, as many as 10⁴ cancer cells/ml blood circulate in this group of metastatic cancer patients. The melting curve analysis of PCR products revealed a surprising result for CEA. All samples of the control and cancer patients groups showed a CEA product at 79° C. melting temperature instead of the expected 82° C. However, eight of cancer patients (26%) had in addition the 82° C. product (FIG. 2A).

Spiking of tumor cells in whole blood. To further study this phenomenon which indicates the presence of a different CEA-like product in white blood cells we spiked LS147T cells in 10 ml whole blood of a healthy volunteer using 10-fold dilution from 10⁷ cells to 1 cell. From this we determined the melting point of the products of real-time RT-PCR. As shown in FIG. 2B we found a major peak at 79° C. but with increasing numbers of tumor cells the peak shifted stepwise to 82° C. and completely replaced the 79° C. peak when the tumor cells exceeded the number of white blood cells. This data strongly suggest that a competing target cDNA is present in white blood cells which interferes with measurement of the tumor cell CEA cDNA (lower panel).

Sequence analysis. We sequenced the real-time PCR products from three different control blood samples, 3 cancer patients' samples which showed both products, and from human HT29 and LS147T colon cancer cells. FIG. 3A gives an overview of the CEA gene and the derived CEA cDNA (FIG. 3B +3 C). The sequence of the PCR product from both cancer cell lines and the CEA cDNA control plasmid were homologue with the expected sequence (FIG. 3B). In contrast, all three samples from the control group and 2 of the cancer group resulted in a product which is composed by a shorter part of the M exon, followed by intron sequence starting shortly before the last 3′ exon which is transcribed in the same way as the tumor cell CEA cDNA product (FIG. 3C). Blast search (National Center for Biotechnology Information, NCBI) did not reveal any known corresponding protein.

The sequence of the PCR product of one cancer patient with the highest 82° C. peak corresponded to the tumor cell CEA cDNA sequence.

The purpose of this study was to establish real-time RT-PCR using CEA and CK20 as detection markers for quantification of circulating tumor cells in whole blood of colon cancer patients. Previous studies indicate that both markers may serve as specific tumor cell markers in the analysis of whole blood samples (37, 38, 42-44). However, according to our experience and reports from others (43, 45, 46) low CEA and CK20 levels are also present in normal blood samples. Thereby, standardization becomes difficult between various experiments and certainly between different research groups because a positive signal depends highly on minor assay condition in relation to cycle numbers. Quantification by real-time RT-PCR should circumvent the problem of “false positive” signals by measuring background levels and defining cut-off values. Furthermore, this approach allows convenient follow-up measurments and, thus, may become an useful marker for therapeutic monitoring.

In the first series of experiments we compared the Amplifluor® and SYBR®-Green methods. The Amplifluor® approach was less sensitive and achieved only significant signals in patients samples when used in a nested-PCR approach. Because a nested approach significantly altered the quality of the standard curve, reliable quantification was not possible. Because of its high sensitivity, SYBR®-Green was the staining method of choice. The higher risk of measuring unspecific fluorescence signals was controlled by melting curve analysis of each sample. Anyway, unspecific products did not appear in this study.

To demonstrate the ability of the method to distinguish between cancer and non-cancer we chose two extreme groups, i.e. metastatic colon cancer patients and healthy volunteers. A method which would not demonstrate differences in blood levels of these markers between these groups obviously would be of little clinical benefit.

We found CK20 to be a useful marker to define cancer cells because 36% of cancer patients showed at least 100-fold higher CK20 values compared to the highest value in the healthy control group. This finding is in accordance with studies using conventional nested RT-PCR, which demonstrated in stage IV colon cancer patients approximately 40% CK20 positivity of whole blood samples (44). However, our study also clearly shows that CK20, an “epithelial cell marker”, is expressed at low levels by mesenchymal white blood cells and underscrores the importance of signal quantification. Assuming that HT29 colon cancer cells and circulating cancer cells in patients have similar CK20 levels, we can roughly estimate that up to 10⁴ cancer cells/ml blood circulate in metastatic cancer patients.

Only few clinical studies have analyzed the usefulness of CEA for detection of circulating colon cancer cells in whole blood samples and none of them have quantified the PCR product by real-time RT-PCR. Recently, a large study was published which applied the primers used in our study to determine CEA mRNA level in whole blood cells before and after surgical therapy (47). In contrast to our study, CEA nested RT-PCR revealed a high number of CEA positive cancer patients but did not demonstrate a CEA signal in the healthy control group. It remains unclear why over a long postoperative period blood samples remained positive despite curative surgery. Other groups detected CEA in a significant number of normal blood samples (13) or in patients with inflammatory bowel disease (48) positive for CEA. It was also found that treatment with GM-CSF induced CEA expression in white blood cells (49). In our study, there was only 10-fold increase in the signal of cancer patients compared to the control group. Only two patients (6%) were responsible for this small elevation of the mean value.

Further analysis revealed that normal white blood cells express a splice variant of CEA in which an “intron” sequence replaces part of the exon. This finding is highly surprising because such a splice variant has not been described in white blood cells yet. In addition to the splice variant, we found cancer patients also had the “expected” CEA product. A spiking experiment with tumor cells mimicked the results of the melting curve analysis of cancer patients indicating that the “cancer cell product” competes with a different product in white blood cells. Most recently, we confirmed the presence of the splice variant using a primer, which binds to the intron sequence a few nucleotide before the 3′ exon starts. The combination of this primer with primer C produced the same sequence of the splice variant (data not shown).

Overall, our data help to explain why “background signals” have been frequently reported (46) despite the lack of CEA protein-expression by white blood cells. Furthermore, this finding may help to redesign the PCR-primer to obtain higher specificity of CEA RT-PCR.

It will be very interesting to define the corresponding protein. CEA is the most widely known tumor marker in colon cancer and high expression levels have been correlated with poor survival (50,51). CEA contributes to the metastatic capacity of colon cancer cells by an adhesive function (52, 53) and, as shown most recently, by protecting tumor cells against apoptosis (54, 55). Our findings may help to better understand its role in tumor biology. It is an interesting hypothesis that the CEA splice variant represents a physiological product, which is distinct from CEA expressed in malignancy. Clearly, further studies are needed to support this speculation which is supported by the fact that CEA research has exclusively focused on cancer cell CEA. For example, the determination of the Exon/Intron sequence has been derived from human colon cancer cells (56).

In summary, is based on the discovery that RT-PCR for CEA in whole blood samples is altered by a splice variant in white blood cells resulting in a high background. The invention provides new diagnosis tools that reduce or totally eliminate the background created by the detection of the splice by targeting the “non-spliced” CEA as a true indicator and quantitator of cancerous cells.

### REFERENCES

1. Mettlin, C. Recent developments in colorectal cancer epidemiology and early detection. Curr. Opinion Oncol., 2:731-737, 1990.
2. Isacoff, W. H., Borud K. H. Chemotherapy for the treatment of patients with metastatic colorectal cancer. World J. Surg. 21:748-762, 1997.
3. Jessup, J. M., and Thomas, P. CEA and metastasis: a facilitator of site-specific metastasis. In: Stanners, C. P. (ed.) Cell adhesion and comunication mediated by the CEA family, pp.195-222. Amsterdam: Harwood Academic Publishers, 1998.
4. Kos, J., Nielsen, H.-J., Krasovec, M., Christensen, I., Cimerman, N., Stephens, R., Brünner, N. Prognostic values of cathepsin B and carcinoembryonic antigen in sera of patients with colorectal cancer. Clin, Cancer Res., 4:1511-1516, 1998.
5. Nakane, Y., Okamura, S., Akehira, K., Boku, T., Okusa, T., Tanaka, K., Hioki, K. Correlation of preoperative carcinoembryonic antigen levels and prognosis of gastric cancer patients. Cancer, 73:2703-2708, 1994.
6. Vinolas, N., Molina, R., Galan, M. C., Casas, F., Callejas, M., Filella, X., Grau, J., Ballesta, A., Estape, J. Tumor markers in response monitoring and prognosis of non small cell lung cancer: preliminary report. Anticancer Res., 18:631-634, 1998.
7. Ebeling, F., Schmitt, U., Untch Michael. Nagel D., Fateh-Moghadam A., Stieber P., Seidel D. Tumor markers CEA and CA 15-3 as prognostic factors in breast cancer—univariate and multivariate analysis. Anticancer Res., 19:2545-2550, 1999.
8. Tokunaga, N., Kijima, H., Noto, T., Osamura, Y., Sadahiro, S., Tajima, T., Mitomi, T. Imunohistochemical localization of carcinoembryonic antigen as a predictor of lymph node status in submucosa-invasive colorectal carcinoma. Dis. Colon Rectum., 38:842-847, 1995.
9. Vogel, I., Francksen, H., Soeth, E., Henne-Bruns, D., Kremer, B., and Juhl, H. The carcinoembryonic antigen (CEA) and its prognostic impact on immunocytologically detected intraperitoneal colorectal cancer cells. Am. J. Surg., in press.
10. Gold, P., and Freedman, S. Specific carcinoembryonic antigens of the human digestive system. J. Exp. Med., 122:467-481, 1965.
11. Hammarstrom, S. The carcinoembryonic antigen (CEA) family:structures, suggested functions and expression in normal and malignant tissues. Semin. Cancer Biol., 2:67-81, 1999.
12. Neumaier, M., Paululat, S., Chan, A., Mafthaes, P., and Wagener, C. Biliary glycoprotein, a potential human cell adhesion molecule, is down-regulated in colorectal carcinomas. Proc. Natl. Acad. Sci. USA., 90:10744-10748, 1993.
13. Benchimol, S., Fuks, A., Jothy, S., Beauchemin, N., Shirota, K., Stanners, C. Carcinoembryonic antigen, a human tumor marker, function as an intercellular adhesion molecule. Cell, 57:327-334, 1989.
14. Stanners, C. P., and Fuks, A. 1998. Properties of adhesion mediated by the human CEA families. In: Stanners, C. P. (ed.) Cell adhesion and comunication mediated by the CEA family, pp. 57-71. Amsterdam: Harwood Academic Publishers, 1998.
15. Zhou, H., Fuks, A., Alcaraz, G., Bolling, T. J., and Stanners, C. P. Homophilic adhesion between lg superfamily carcinoembryonic antigen molecules involves double reciprocal bonds. J. Cell Biol., 122:951-960, 1993.
16. Taheri, M., Saragovi, U., Fuks, A., Makkerh, J., Mort, J., Stanners, C. P. Self recognition in the lg superfamily. J. Biol. Chem. 275:26935-26943, 2000.
17. Edmiston, K., Gangopadhyay, A., Shoji, Y., Nachman, A., Thomas, P., Jessup, J. M. In vivo induction of murine cytokine production by carcinoembryonic antigen. Cancer Res., 57:4432-4436, 1997.
18. Bayon, L., lzquierdo, M., Sirovich, I., van Rooijen, N., Beelen, R., and Meijer, S. Role of Kupfer cells in arresting circulating tumor cells and controlling metastatic growth in the liver. Hepatology, 23:1224-1231, 1996.
19. Von Kleist, S., Migule, I, and Halla, B. Possible function of CEA as cell-contact inhibitory molecule. Anticancer Res., 15:1889-1894, 1995.
20. Kammerer, R., and v. Kleist, S. The carcinoembryonic antigen (CEA) modulates effector-target cell interaction by binding to activated lymphocytes. Int. J. Cancer., 68:457-463, 1996.
21 .Gangopadhyay, A., Lazure, D., and Thomas, P. Adhesion of colorectal carcinoma cells to the endothelium is mediated by cytokines from CEA stimulated Kupffer cells. Clin. Exp. Metastasis, 16:703-712, 1998.
22. Takahashi, H., Okai, Y., Paxton, R., Hefta, L., Shively, J. Differential regulation of carcinoembryonic antigen and biliary glycoprotein by (−interferon. Cancer Res. 53:1612-1619, 1993.
23. Gossen, M., and Bujard, H. Tight control of gene expression in mammalian cells by tetracycline-responsive promoters. Proc. Natl. Acad. Sci. USA, 89:5547-5551, 1992.
24. Schulte, A., Lai, S., Kurtz, A., Czubayko, F., Riegel, A. T., and Wellstein, A. Human trophoblast and choriocarcinoma expression of the growth factor pleiotrophin attributable to germ-line insertion of an endogenous retrovirus. Proc. Natl. Acad. Sci. USA., 93:14759-14764, 1996.
25. Juhl, H., Downing, S., Wellstein, A., and Czubayko, F. HER-2/neu rate limiting for ovarian cancer growth: conditional depletion of HER-2/neu by ribozyme-targeting. J. Biol. Chem., 272:29482-29486, 1997.
26. Palfner, K., Kneba, M., Hiddemann, W., and Bertram, J. Short technical reports. Quantification of ribozyme-mediated RNA cleavage using silver-stained polyacrylamide gels. Biotechniques, 19 (6):926-91995, 1995.
27. Fang, W. J., Hartmann, N., Chow, D., Riegel, A. T., and Wellstein, A. Pleiotrophin stimulates fibroblasts and endothelial and epithelial cells and is expressed in human cancer. J. Biol. Chem., 267:25889-25897, 1992.
28. Vindelov, L. L., Christensen, l. J., Nissen, N.I. Standardization of high-resolution flow cytometric DNA analysis by the simultaneous use of chicken and trout red blood cells as internal reference standards. Cytometry, 3:323-327, 1983.
29. Freeman, W. E., Robertson, D. J., Vrana, K. E. Fundamentals of DNA hybridization arrays for gene expression analysis. BibTechniques 29:1042-1055, 2000.
30. O'Connell, J., Bennett, M. W., Nally, K., O'Sullivan, G. C., Collins, J. K., Shanahan, F. Interferon-gamma sensitizes colonic epithelial cell lines to physiological and therapeutic inducers of colonocyte apoptosis. J. Cell Physiol., 185(3):331-338, 2000.
31. Nita, M. E., Nagawa, H., Tominaga, O., Tsuno, N., Fujii, S., Sasaki, S., Fu, C. G., Takenoue, T., Tsuruo, T., Muto, T. 5-Fluorouracil induces apoptosis in human colon cancer cell lines with modulation of Bcl-2 family proteins. Br. J. Cancer, 78(8):986-92, 1998.
32. Wemer, M., and Uhlenbeck, O. The effect of base mismatches in the substrate recognition helices of hammerhead ribozymes on binding and catalysis. Nucleic Acids Res., 23:2092-2096, 1995.
33. Engell, H. Cancer cells in the circulating blood., Acta Chir. Scand. Suppl. 210: 10-70, 1955.
34. Redding, W. H., Coombes, R. C., Monaghan, P., Clink, H. M., Imrie, S. F., Dearnaley, D. P., Ormerod, M. G., Sloane, J. P., Gazet, J. C., Powles, T. J., and et al. Detection of micrometastases in patients with primary breast cancer, Lancet. 2: 1271-4., 1983.
35. Schlimok, G., Funke, I., Holzmann, B., Gottlinger, G., Schmidt, G., Hauser, H., Swierkot, S., Warnecke, H. H., Schneider, B., Koprowski, H., and et al. Micrometastatic cancer cells in bone marrow: in vitro detection with anti-cytokeratin and in vivo labeling with anti-17-1A monoclonal antibodies, Proc Natl Acad Sci U S A. 84: 8672-6., 1987.
36. Juhl, H., Stritzel, M., Wroblewski, A., Henne-Bruns, D., Kremer, B., Schmiegel, W., Neumaier, M., Wagener, C., Schreiber, H. W., and Kalthoff, H. Immunocytological detection of micrometastatic cells: comparative evaluation of findings in the peritoneal cavity and the bone marrow of gastric, colorectal and pancreatic cancer patients, Int J Cancer. 57: 330-5., 1994.
37. Gerhard, M., Juhl, H., Kalthoff, H., Schreiber, H. W., Wagener, C., and Neumaier, M. Specific detection of carcinoembryonic antigen-expressing tumor cells in bone marrow aspirates by polymerase chain reaction, J Clin Oncol. 12: 725-9., 1994.
38. Burchill, S. A., Bradbury, M. F., Pittman, K., Southgate, J., Smith, B., and Selby, P. Detection of epithelial cancer cells in peripheral blood by reverse transcriptase-polymerase chain reaction, Br J Cancer. 71: 278-81., 1995.
39. Soeth, E., Roder, C., Juhl, H., Kruger, U., Kremer, B., and Kalthoff, H. The detection of disseminated tumor cells in bone marrow from colorectal-cancer patients by a cytokeratin-20-specific nested reverse-transcriptase-polymerase-chain reaction is related to the stage of disease, Int J Cancer. 69: 278-82., 1996.
40. Lindemann, F., Schlimok, G., Dirschedl, P., Witte, J., and Riethmuller, G. Prognostic significance of micrometastatic tumour cells in bone marrow of colorectal cancer patients, Lancet. 340: 685-9., 1992.
41. Schott, A., Vogel, I., Krueger, U., Kalthoff, H., Schreiber, H. W., Schmiegel, W., Henne-Bruns, D., Kremer, B., and Juhl, H. Isolated tumor cells are frequently detectable in the peritoneal cavity of gastric and colorectal cancer patients and serve as a new prognostic marker, Ann Surg. 227: 372-9., 1998.
42. Mori, M., Mimori, K., Ueo, H., Karimine, N., Barnard, G. F., Sugimachi, K., and Akiyoshi, T. Molecular detection of circulating solid carcinoma cells in the peripheral blood: the concept of early systemic disease, Int J Cancer. 68: 73943., 1996.
43. Liefers, G. J., Cleton-Jansen, A. M., van de Velde, C. J., Hermans, J., van Krieken, J. H., Cornelisse, C. J., and Tollenaar, R. A. Micrometastases and survival in stage II colorectal cancer, N Engl J Med. 339: 223-8., 1998.
44. Soeth, E., Vogel, I., Roder, C., Juhl, H., Marxsen, J., Kruger, U., Henne-Bruns, D., Kremer, B., and Kalthoff, H. Comparative analysis of bone marrow and venous blood isolates from gastrointestinal cancer patients for the detection of disseminated tumor cells using reverse transcription PCR, Cancer Res. 57: 3106-10., 1997.
45. Ko, Y., Klinz, M., Totzke, G., Gouni-Berthold, I., Sachinidis, A., and Vetter, H. Limitations of the reverse transcription-polymerase chain reaction method for the detection of carcinoembryonic antigen-positive tumor cells in peripheral blood, Clin Cancer Res. 4: 2141-6., 1998.
46. Goeminne, J. C., Guillaume, T., and Symann, M. Pitfalls in the detection of disseminated non-hematological tumor cells, Ann Oncol. 11: 785-92., 2000.
47. Guadagni, F., Kantor, J., Aloe, S., Carone, M. D., Spila, A., D'Alessandro, R., Abbolito, M. R., Cosimelli, M., Graziano, F., Carboni, F., Carlini, S., Perri, P., Sciarretta, F., Greiner, J. W., Kashmiri, S. V., Steinberg, S. M., Roselli, M., and Schlom, J. Detection of blood-borne cells in colorectal cancer patients by nested reverse transcription-polymerase chain reaction for carcinoembryonic antigen messenger RNA: longitudinal analyses and demonstration of its potential importance as an adjunct to multiple serum markers, Cancer Res. 61: 2523-32., 2001.
48. Castells, A., Boix, L., Bessa, X., Gargallo, L., and Pique, J. M. Detection of colonic cells in peripheral blood of colorectal cancer patients by means of reverse transcriptase and polymerase chain reaction, Br J Cancer. 78: 1368-72., 1998.
49. Jung, R., Kruger, W., Hosch, S., Holweg, M., Kroger, N., Gutensohn, K., Wagener, C., Neumaier, M., and Zander, A. R. Specificity of reverse transcriptase polymerase chain reaction assays designed for the detection of circulating cancer cells is influenced by cytokines in vivo and in vitro, Br J Cancer. 78: 1194-8., 1998.
50. Grem, J. The prognostic importance of tumor markers in adenocarcinomas of the gastrointestinal tract, Curr Opin Oncol. 9: 380-7., 1997.
51. Vogel, I., Francksen, H., Soeth, E., Henne-Bruns, D., Kremer, B., and Juhl, H. The carcinoembryonic antigen and its prognostic impact on immunocytologically detected intraperitoneal colorectal cancer cells, Am J Surg. 181: 188-93., 2001.
52. Benchimol, S., Fuks, A., Jothy, S., Beauchemin, N., Shirota, K., and Stanners, C. P. Carcinoembryonic antigen, a human tumor marker, functions as an intercellular adhesion molecule, Cell. 57: 327-34., 1989.
53. Gangopadhyay, A., Lazure, D. A., and Thomas, P. Adhesion of colorectal carcinoma cells to the endothelium is mediated by cytokines from CEA stimulated Kupffer cells, Clin Exp Metastasis. 16: 703-12., 1998.
54. Soeth, E., Wirth, T., List, H. J., Kumbhani, S., Petersen, A., Neumaier, M., Czubayko, F., and Juhl, H. Controlled Ribozyme Targeting Demonstrates an Antiapoptotic Effect of Carcinoembryonic Antigen in HT29 Colon Cancer Cells, Clin Cancer Res. 7: 2022-30., 2001.
55. Wirth, T., Soeth, E., Czubayko, F., and Juhl, H. Inhibition of endogenous Carcinoembryonic Antigen (CEA) increases the apoptotic rate of colon cancer cells and inhibits metastatic tumor growth, Clin. Exp. Metastasis, in press.
56. Schrewe, H., Thompson, J., Bona, M., Hefta, L. J., Maruya, A., Hassauer, M., Shively, J. E., von Kleist, S., and Zimmermann, W. Cloning of the complete gene for carcinoembryonic antigen: analysis of its promoter indicates a region conveying cell type-specific expression, Mol Cell Biol. 10: 2738-48., 1990.

What is claimed is:

1. A method of detecting carcinoembryonic antigen (CEA) comprising detecting a DNA sequence comprising the following sequence:
GATATAGCAGCCCTGGTGTAGTTTCTTCATTTCAGGAAGACTG.

2. A method of diagnosis of disseminated cancer comprising obtaining a cell sample, extracting DNA from the cell and detecting the presence of the following sequence:
GATATAGCAGCCCTGGTGTAGTTTCTTCATTTCAGGAAGACTG.

3. The method of claim 2, wherein detecting the sequence comprises conducting RT PCR.

4. The method of claim 3, wherein conducting RT PCR comprises designing a primer comprising the complementary of the sequence or a portion thereof.

5. An isolated nucleic acid molecule comprising a polynucleotide selected from the group consisting of:
(e) the sequence defined by nucleotide 49 to nucleotide 108 contained in FIG. 4C;
(f) the sequence defined by nucleotide 50 to nucleotide 107 contained in FIG. 4C;
(g) the polynucleotide complement of the polynucleotide of (a) or (b);
and
(h) a polynucleotide at least 90% identical to the polynucleotide of (a), (b) or (c).

6. An isolated nucleic acid molecule comprising about 5 to about 60 contiguous nucleotides from the sequence defined by nucleotide 49 to nucleotide 108 contained in FIG. 4C.

7. The isolated nucleic acid molecule of claim 5, which is DNA.

8. A method of making a recombinant vector comprising inserting a nucleic acid molecule of claim 5 into a vector in operable linkage to a promoter.

9. A recombinant vector produced by the method of claim 8.

10. A method of making a recombinant host cell comprising introducing the recombinant vector of claim 9 into a host cell.

13. A recombinant host cell produced by the method of claim 10.

12. A recombinant method of producing a polypeptide, comprising culturing the recombinant host cell of claim 11 under conditions such that said polypeptide is expressed and recovering said polypeptide.

13. An isolated polypeptide comprising amino acids at least 95% identical to amino acids encoded by a polynucleotide selected from the group consisting of:
(a) the sequence defined by nucleotide 49 to nucleotide 108 contained in FIG. 4C;
(b) the sequence defined by nucleotide 50 to nucleotide 107 contained in FIG. 4C;
(c) the polynucleotide complement of the polynucleotide of (a) or (b); and
(d) a polynucleotide at least 90% identical to the polynucleotide of (a), (b) or (c).

14. An isolated polypeptide wherein, except for at least one conservative amino acid substitution, said polypeptide has an amino acid sequence encoded by a polynucleotide selected from the group consisting of:
(a) the sequence defined by nucleotide 49 to nucleotide 108 contained in FIG. 4C;
(b) the sequence defined by nucleotide 50 to nucleotide 107 contained in FIG. 4C;
(c) the polynucleotide complement of the polynucleotide of (a) or (b); and
(d) a polynucleotide at least 90% identical to the polynucleotide of (a), (b) or (c).

15. An isolated polypeptide comprising amino acids encoded by 5 to 60 contiguous nucleotides selected from the group consisting of:
(a) the sequence defined by nucleotide 49 to nucleotide 108 contained in FIG. 4C;
(b) the sequence defined by nucleotide 50 to nucleotide 107 contained in FIG. 4C;
(c) the polynucleotide complement of the polynucleotide of (a) or (b);
and
a polynucleotide at least 90% identical to the polynucleotide of (a), (b) or (c).

16. An epitope-bearing portion of a polypeptide encoded by a nucleic acid sequence selected from the group consisting of:
(a) the sequence defined by nucleotide 49 to nucleotide 108 contained in FIG. 4C;
(b) the sequence defined by nucleotide 50 to nucleotide 107 contained in FIG. 4C;
(c) the polynucleotide complement of the polynucleotide of (a) or (b);
and
(d) a polynucleotide at least 90% identical to the polynucleotide of (a), (b) or (c).

17. The epitope-bearing portion of claim 16, which comprises about 5 to about 30 contiguous amino acids encoded by a nucleic acid sequence selected from the group consisting of:
(a) the sequence defined by nucleotide 49 to nucleotide 108 contained in FIG. 4C;
(b) the sequence defined by nucleotide 50 to nucleotide 107 contained in FIG. 4C;
(c) the polynucleotide complement of the polynucleotide of (a) or (b); and
(d) a polynucleotide at least 90% identical to the polynucleotide of (a), (b) or (c).

18. An isolated antibody that binds specifically to the polypeptide of claim 15.

19. A monoclonal antibody according to claim 18.
